(19) United States
(12) Patent Application Publication
(10) Pub. No.: US 2008/0015658 A1
(43) Pub. Date: Jan. 17, 2008

(54) Sound sensed electric stimulation control device

(76) Inventor: Chao-Man Tseng, Taichung (TW)

Correspondence Address: Chao-Man Tseng, 235 Chung-Ho, Box 8-24, Taipei, omitted

(21) Appl. No.: 11/484,301

(22) Filed: Jul. 12, 2006

(51) Int. Cl. A61N 1/18

(52) U.S. Cl. 607/62; 607/55

(57) ABSTRACT

A sound sensed electric stimulation control device comprises a body. The body further comprises a signal analysis unit for receiving sound signals and converting the signals into output signals; and an electronic stimulation output portion for generating corresponding output energy according to the output signals. The output emerge is controlled by the inputted sound signals to the signal analysis unit. The signal analysis unit is connected to a signal input unit. The signal analysis unit is connected to a sound input unit. The signal analysis unit is connected to a built-in sound input unit. The signal analysis unit includes a frequency band energy detector, a detected energy identifier and an electronic stimulation control unit so as to analyze features of inputted sound signals and then convert the sound signals into output signals so as to stimulate the electronic stimulation output portion to output energy.

### FIELD OF THE INVENTION

The present invention relates to a sound sensed electric stimulation control device which has various input operation modes instead of only one operation mode of built-in sound signals as those in the prior art.

### BACKGROUND OF THE INVENTION

Electronic stimulations have widely used to the human bodies for medical reasons.

Current electronic stimulation device such as TENS (transcutaneous electric nerve stimulator), EMS (electric muscle stimulator), etc. provides many medical functions, such as relieve-pain, physical treatment, exercise, warm-up exercise, body shaping, recovering, etc. Thus they are widely used in medical institutions. Thereby the stimulators are sold widely in the markets so that users can operate the electronic stimulators by himself (or herself).

The prior art electronic stimulator has built in stimulation modes and a plurality of buttons are provided so that the users can adjust the energies and frequencies. The stimulations are performed periodically according to the frequencies and energies.

However the prior art electronic stimulators only have built in operation modes. They can not be operated by other non-built in modes. Thereby the operation is dull. Generally, the electronic stimulator must be used for a long time so as to have a desired affect, while above mentioned defect makes the user fee uneasy and not to use these devices. The electronic stimulation must be used for a long time so as to have a desired effect. If the users cannot use the devices continuously, the effect is not preferable. As a whole, the prior art structure is not practical and thus is necessary to be improved.

### SUMMARY OF THE INVENTION

Accordingly, the primary object of the present invention is to provide a sound sensed electric stimulation control device which has various input operation modes instead of only one operation mode of built—in sound signals as those in the prior art.

To achieve above objects, the present invention provides a sound sensed electric stimulation control device which comprises a body. The body further comprises a signal analysis unit for receiving sound signals and converting the signals into output signals; and an electronic stimulation output portion for generating corresponding output energy according to the output signals. The output emerge is controlled by the inputted sound signals to the signal analysis unit. The signal analysis unit is connected to a signal input unit. The signal analysis unit is connected to a sound input unit. The signal analysis unit is connected to a built-in sound input unit. The signal analysis unit includes a frequency band energy detector, a detected energy identifier and an electronic stimulation control unit so as to analyze features of inputted sound signals and then convert the sound signals into output signals so as to stimulate the electronic stimulation output portion to output energy.

The various objects and advantages of the present invention will be more readily understood from the following detailed description when read in conjunction with the appended drawing.

### BRIEF DESCRIPTION OF THE DRAWINGS

FIG. 1 is a block diagram of the sound sensed electric stimulation control device of the present invention.

FIG. 2 is a schematic perspective view of the sound sensed electric stimulation control device of the present invention.

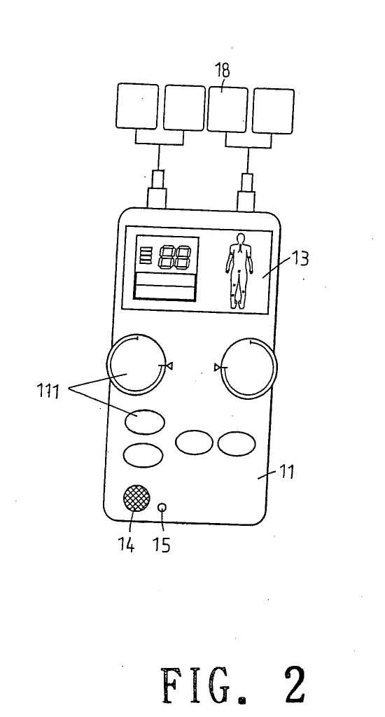

FIG. 3 is a schematic view showing the built in mode in the sound sensed electric stimulation control device of the present invention.

FIG. 4 is a schematic view showing the use of the sound input mode according to the present invention.

FIG. 5 is a schematic view showing the use of the signal input mode of the sound sensed electric stimulation control device of the present invention.

FIG. 6 is a schematic view, where the present is built with a sound signal module in the sound sensed electric stimulation control device.

### DETAILED DESCRIPTION OF THE INVENTION

In order that those skilled in the art can further understand the present invention, a description will be described in the following in details. However, these descriptions and the appended drawings are only used to cause those skilled in the art to understand the objects, features, and characteristics of the present invention, but not to be used to confine the scope and spirit of the present invention defined in the appended claims.

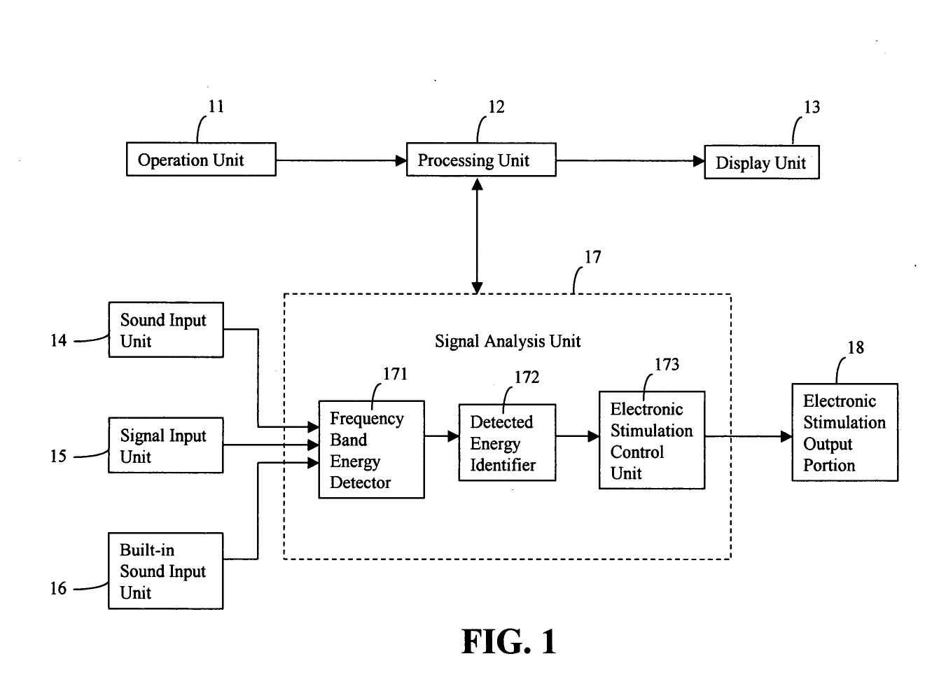

Referring to FIG. 1, the sound sensed electric stimulation control device of the present invention is illustrated. The present invention has the following elements.

A body 1 is included.

An operation unit 11 includes a plurality of buttons 111 and is connected to a processing unit 12 in the body 1. Thereby a user can select a desired mode through the operation unit 11. The modes include a built-in mode, a sound input mode, a signal input mode, a built-in sound input mode. The operation unit 11 transfers a selected mode to a signal analysis unit 17. The related information about the mode is displayed through a display unit 13.

The signal analysis unit 17 is installed in the body 1. The current operation mode is adjusted according to the operation unit 11 and the processing unit 12. The signal analysis unit 17 includes a frequency band energy detector 171, a detected energy identifier 172 and an electronic stimulation control unit 173.

The frequency band energy detector 171 is connected to a sound input unit 14, a signal input unit 15, and a built-in sound input unit 16. The sound input unit 14 is for example a sound transmitter by which a user can transfer sound to the frequency band energy detector 171 of the signal analysis unit 17 by sounds. The signal input unit 15 is a connection port which can be connected to other electron device stored with sound messages so that the user can input sound signals to the frequency band energy detector 171 of the signal analysis unit 17. The built-in sound input unit 16 can be built with a plurality of different sound signals for being selected by users so that the built-in sound signals can be inputted to the frequency band energy detector 171 of the signal analysis unit 17. Preferably, the sound signals are music or other harmonic sound signals.

When the sound signals are transferred to the signal analysis unit 17, the frequency band energy detector 171 gets the energy of the frequency band through a digital filter or an analog filter. Then the detected energy identifier 172 serves the identify and the energy of the frequency band and then coverts the signals into output signals. Then the converted signals are outputted to an electronic stimulation output portion 18 through the electronic stimulation control unit 173. Then the electronic stimulation output portion 18 generates corresponding output energies according to the output signals so as to provide stimulation to the user's skins or muscles.

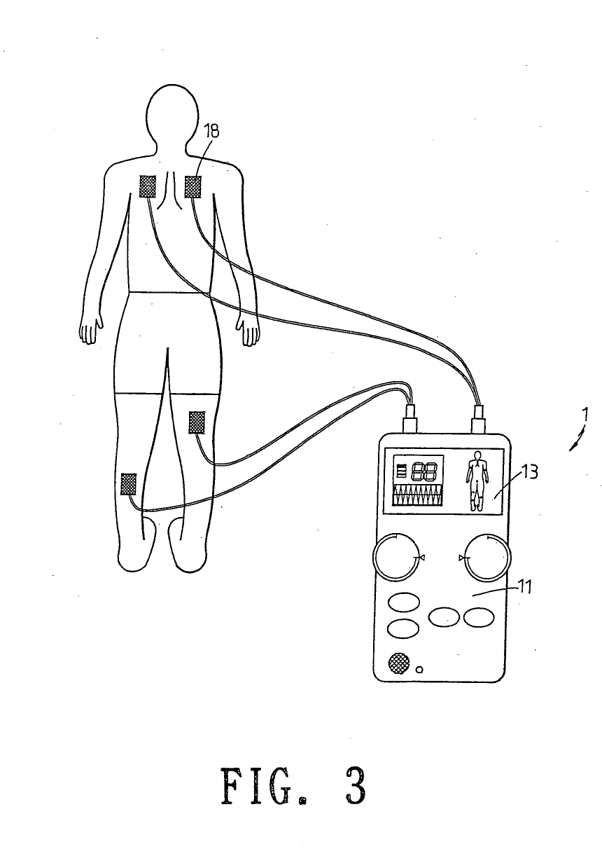

In use of the present invention, referring to FIGS. 3 to 6, the electronic stimulation output portion 18 are placed upon the skin of the user, and the operation unit 11 is adjusted to a desired operation mode. The display unit 13 displays current mode or class through indicators or waves of output energies.

Referring to FIG. 3, when the user adjusts the built in modes through the operation unit 11, the user adjusts necessary frequency and strength through the operation unit 11. Then by the operation processing unit 12 and the signal analysis unit 17, the electronic stimulation output portion 18 generates frequencies, strengths and energies requested by users so as to provide electronic stimulation to users.

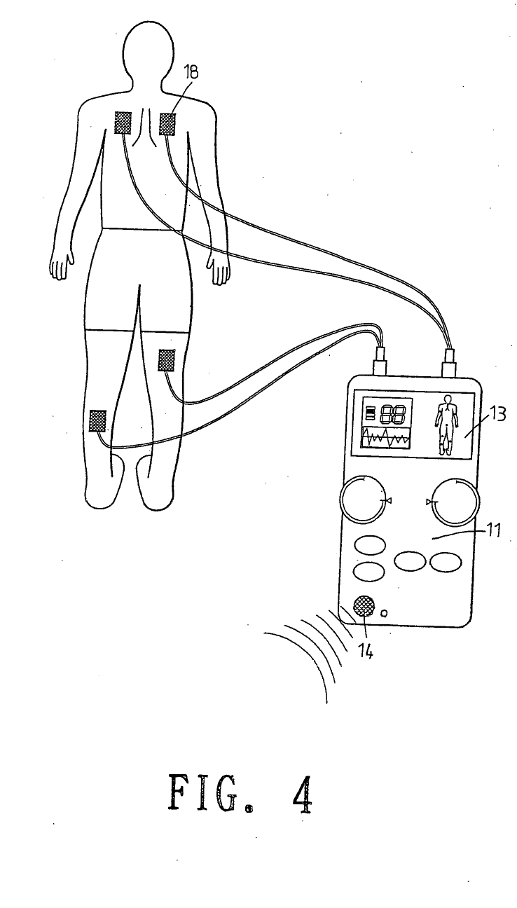

Referring to FIG. 4, when the user adjusts the sound input mode through the operation unit 11, the user can input the sound signals to the signal analysis unit 17 through the sound input unit 14 so that the sound signals are directly inputted to the signal analysis unit 17 and then the sound signals are converted into output signals through the frequency band energy detector 171, detected energy identifier 172 and electronic stimulation control unit 173 to control the electronic stimulation output portion 18 to generate desired energy for stimulating the users.

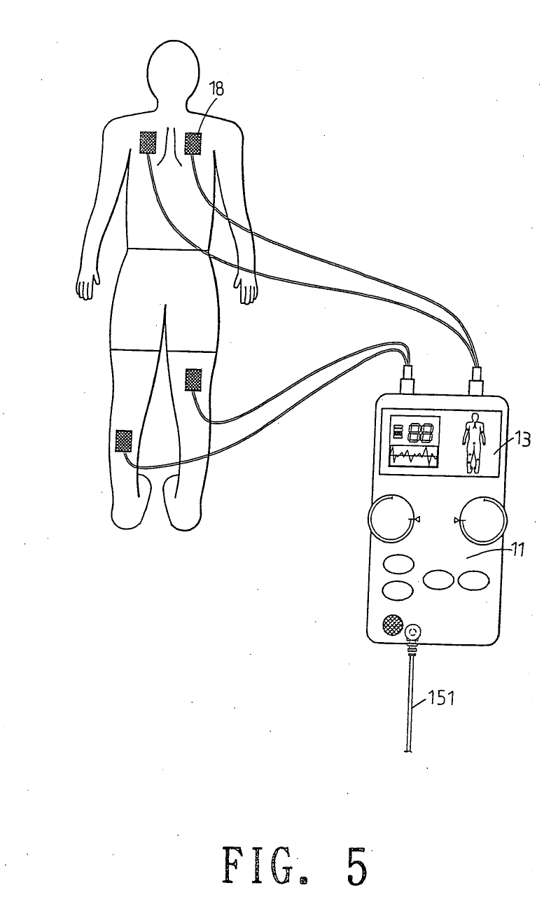

Referring to FIG. 5, when the user adjusts the signal input mode through the operation unit 11, the user can connect a sound stored electronic device to a transmission line or the signal input unit 15. The electronic device inputs sound signals to the signal analysis unit 17 and then the sound signals are converted into output signals through the frequency band energy detector 171, detected energy identifier 172 and electronic stimulation control unit 173 to control the electronic stimulation output portion 18 to generate desired energy for stimulating the users.

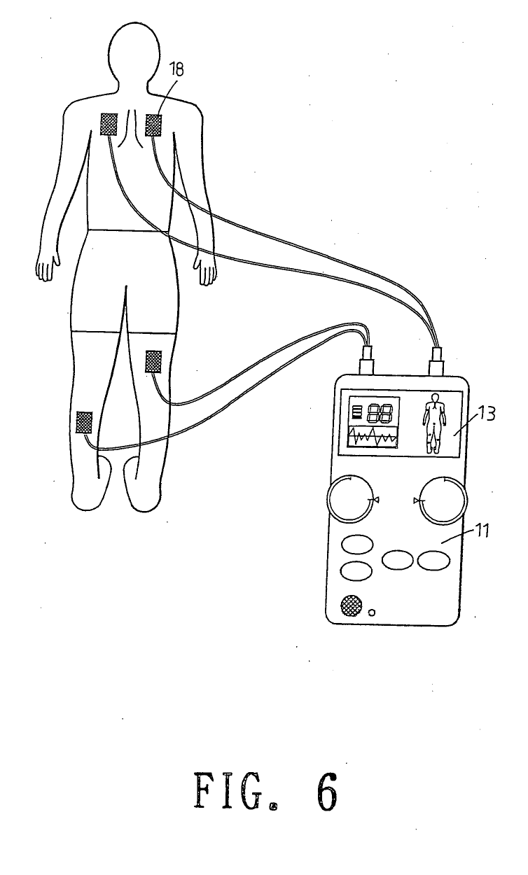

With reference to FIG. 6, when the user adjusts the built-in sound input unit 16 through the operation unit 11, the user can select the built in sound signals stored in the built-in sound input unit 16 so that the built-in sound input unit 16 inputs signals to the signal analysis unit 17 and then the sound signals are converted into output signals through the frequency band energy detector 171, detected energy identifier 172 and electronic stimulation control unit 173 to control the electronic stimulation output portion 18 to generate desired energy for stimulating the users.

By above mentioned structure and way, the user can select desired input sounds to control the output stimulation energy of the body 1.

However in the present invention, other than the frequency band energy detector 171, detected energy identifier 172, and electronic stimulation control unit 173, the signal analysis unit 17 may have any desired elements to achieve the object of converting sound signals into output signals. Moreover, the present invention can be realized by software or hardware. All these are within the scope of the present invention.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A sound sensed electric stimulation control device comprising:
a body further comprising:
a signal analysis unit for receiving sound signals and converting the signals into output signals; and
an electronic stimulation output portion for generating corresponding output energy according to the output signals;
whereby the output signals are controlled by the inputted sound signals to the signal analysis unit.

2. The sound sensed electric stimulation control device as claimed in claim 1, wherein the signal analysis unit is connected to a signal input unit.

3. The sound sensed electric stimulation control device as claimed in claim 1, wherein the signal analysis unit is connected to a sound input unit.

4. The sound sensed electric stimulation control device as claimed in claim 1, wherein the signal analysis unit is connected to a built-in sound input unit.

5. The sound sensed electric stimulation control device as claimed in claim 1, wherein the signal analysis unit includes a frequency band energy detector, a detected energy identifier and an electronic stimulation control unit so as to analyze features of inputted sound signals and then convert the sound signals into output signals to stimulate the electronic stimulation output portion to output energy.

6. The sound sensed electric stimulation control device as claimed in claim 1, wherein the sound signals includes direct sound inputs, sound signals input.

7. The sound sensed electric stimulation control device as claimed in claim 1, wherein the body has an operation unit and a processing unit; the operation unit serves for switching operation modes; the processing unit processes the mode selected by the signal analysis unit.

8. The sound sensed electric stimulation control device as claimed in claim 1, wherein the operation unit includes a sound input mode, a signal input mode and a built-in sound input mode.

9. The sound sensed electric stimulation control device as claimed in claim 1, wherein the display unit is connected to a display unit; and the display unit displays message about the operation mode.

10. The sound sensed electric stimulation control device as claimed in claim 1, wherein the display unit displays wave about output energies.
